(19) United States
(12) Patent Application Publication
(10) Pub. No.: US 2011/0195457 A1
(43) Pub. Date: Aug. 11, 2011

(54) ISOTHERMAL AMPLIFICATION OF NUCLEIC ACID USING PRIMERS COMPRISING A RANDOMIZED SEQUENCE AND SPECIFIC PRIMERS AND USES THEREOF

(75) Inventors: John Richard Nelson, Clifton Park, NY (US); Wei Gao, Clifton Park, NY (US); Ming Zhao, Rockville, MD (US)

(73) Assignee: GENERAL ELECTRIC COMPANY, SCHENECTADY, NY (US)

(21) Appl. No.: 12/702,884

(22) Filed: Feb. 9, 2010

(51) Int. Cl. C12P 19/34; C12N 9/12

(52) U.S. Cl. 435/91.2; 435/194

(57) ABSTRACT

Methods and kits for amplifying a nucleic acid under isothermal conditions to form an amplified nucleic acid sequence are provided. The methods and kits comprises providing a nucleic acid template, a DNA polymerase, deoxyribonucleoside triphosphates, a primer comprising a randomized sequence, and a specific primer, and amplifying the nucleic acid template.

### STATEMENT REGARDING FEDERALLY SPONSORED RESEARCH & DEVELOPMENT

This invention was made with Government support under contract number HDTRA1-07-C-0097 awarded by the Defense Threat Reduction Agency. The Government has certain rights in the invention.

### SEQUENCE LISTING

The instant application contains a Sequence Listing, which has been submitted via EFS-Web and is hereby incorporated by reference in its entirety. Said ASCII copy, created on Feb. 3, 2010, is named 237865-1.txt, and is 1,194 bytes in size.

### FIELD OF INVENTION

The invention generally relates to methods and kits for isothermal, strand displacement nucleic acid amplification. The methods specifically relate to isothermal amplification of nucleic acids using a mixture of random primers and specific primers.

### BACKGROUND

A variety of techniques are currently used to amplify nucleic acids, even from a few molecules of a starting nucleic acid template. These include polymerase chain reaction (PCR), ligase chain reaction (LCR), self-sustained sequence replication (3SR), nucleic acid sequence based amplification (NASBA), strand displacement amplification (SDA), multiple displacement amplification (MDA), or rolling circle amplification (RCA).

Nucleic acid amplification techniques are often employed in nucleic acid-based assays used for analyte detection, sensing, forensic and diagnostic applications, genome sequencing, whole-genome amplification, and the like. Such applications often require amplification techniques having high specificity, sensitivity, accuracy, and robustness. The amplification of nucleic acids is particularly important when the starting template nucleic acid is available in minimal amounts. However, most of the currently available techniques for nucleic acid amplification suffer from high background signals, which are generated by non-specific amplification reactions yielding undesired/false amplification products.

Nucleic acid amplification and analysis from a biological sample may be achieved with greater accuracy and ease using isothermal conditions for amplification. The main advantage of isothermal amplification methods over thermal cycling methods (e.g. PCR) is the ability to perform reactions with minimal instrumentation, by avoiding the need for thermal cyclers. The instrumentation for isothermal amplification may use controlled heated blocks or water baths, making the technique more accessible, convenient and economical. Moreover, many of the isothermal amplification techniques such as rolling circle amplification, whole genome amplification, or loop-mediated isothermal amplification (LAMP) may be performed directly with a crude biological material containing target nucleic acids without prior purification of the target nucleic acids. However, in certain specific applications, such as whole-genome amplification, some specific loci of interest may be lost during amplification when the existing amplification methods are employed. So, there is a need to develop better isothermal amplification methods that are designed to preserve all the required sequences that are present in the template nucleic acid.

### BRIEF DESCRIPTION

One or more of the embodiments of the invention provide methods and kits for efficient amplification of nucleic acids. In some embodiments, methods for nucleic acid amplification employing a specific primer and a primer comprising a randomized sequence primers under isothermal condition are provided.

In one embodiment, methods for nucleic acid amplification are provided. The method comprises providing a nucleic acid template, a DNA polymerase, deoxyribonucleoside triphosphates, a primer comprising a randomized sequence, and a specific primer. The method comprises amplifying the nucleic acid template under isothermal conditions to form an amplified nucleic acid sequence.

In another embodiment of the methods for nucleic acid amplification, the method comprises providing a nucleic acid template, a DNA polymerase, deoxyribonucleoside triphosphates, a primer comprising a randomized sequence, and a specific primer. The method comprises amplifying the nucleic acid template under isothermal conditions to form an amplified nucleic acid sequence that is attached to a surface.

In another embodiment, kits for nucleic acid amplification is provided. The kit comprises a Phi29 DNA polymerase; at least one primer comprising a randomized sequence; and at least one specific primer.

### DRAWINGS

These and other features, aspects, and advantages of the present invention will become better understood when the following detailed description is read with reference to the accompanying drawings in which like characters represent like parts throughout the drawings, wherein:

FIG. 1 is a schematic drawing of a rolling circle amplification reaction and capturing of amplified deoxyribonucleic acid (DNA) by bead-bound specific primers, and further amplification of the captured DNA.

FIG. 2 is a schematic drawing of an isothermal rolling circle DNA amplification reaction on a bead showing the transfer of DNA from one bead to other using random primers.

FIG. 3 is a drawing showing the hybridization of a bead-bound DNA to a surface-bound primer and further extension of the surface-bound primer to capture a copy of the DNA on the surface.

FIG. 4A is an image of a single bead coated with amplified DNA and captured DNA. FIG. 4B shows a single bead without any coating of amplified DNA.

FIG. 5 is an image of an agarose-gel illustrating the EcoRI restriction digestion products of amplified pUC 18 DNA captured by a bead-bound specific primer (lane 2) compared to negative and positive controls (lanes 3 and 4).

FIG. 6 is an image of an agarose-gel illustrating the EcoRI restriction digestion products of an amplified DNA captured by a bead-bound specific primer in different conditions.

FIG. 7 is a graph of sequencing data of an amplified DNA (SEQ ID NO: 2) captured on beads.

### DETAILED DESCRIPTION

Nucleic acid-based assays involving single molecule DNA amplification or whole-genome amplification require highly efficient nucleic acid amplification methods that have high yield, high fidelity and little bias in terms of sequence coverage. Isothermal nucleic acid amplification reactions such as rolling circle amplification (RCA), or multiple displacement amplification (MDA) employing primers comprising randomized sequences are more suitable than temperature-dependent nucleic acid amplification reaction (e.g., PCR) for such applications.

One or more embodiments of the invention are directed at methods and kits for efficient isothermal amplification of nucleic acids. In some embodiments, the methods comprise in-vitro amplification of a nucleic acid template that employs two types of primers, one primer comprising a randomized sequence and a specific primer. In some embodiments, the methods comprise in-vitro amplification of a nucleic acid template employing primers comprising a randomized sequence comprising nucleotide nucleic acid amplification reaction. The methods further comprise capturing amplified nucleic acid sequences using specific primers that are attached to a substrate or a capturing agent (alternatively the term “capturing agent” is used herein as a “capture agent”).

To more clearly and concisely describe and point out the subject matter of the claimed invention, the following definitions are provided for specific terms, which are used in the following description and the appended claims. Throughout the specification, exemplification of specific terms should be considered as non-limiting examples.

The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts while still being considered free of the modified term. Where necessary, ranges have been supplied, and those ranges are inclusive of all sub-ranges there between.

As used herein, the term “nucleoside” refers to a glycosylamine compound wherein a nucleic acid base (nucleobase) is linked to a sugar moiety. The nucleic acid base may be a natural nucleobase or a modified/synthetic nucleobase. The nucleic acid base may include, but is not limited to, a purine base (e.g., adenine or guanine), a pyrimidine (e.g., cytosine, uracil, or thymine), or a deazapurine base. The nucleic acid base may be linked to the 1′ position, or at an equivalent position of a pentose (e.g., a ribose or a deoxyribose) sugar moiety. The sugar moiety may include, but is not limited to, a natural sugar, a sugar substitute (e.g., a carbocyclic or an acyclic moiety), a substituted sugar, or a modified sugar (e.g., bicyclic furanose unit as in locked nucleic acid (LNA) nucleotide). The nucleoside may contain a 2′-hydroxyl, 2′-deoxy, or 2′,3′-dideoxy forms of the sugar moiety.

As used herein the terms “nucleotide” or “nucleotide base” refer to a nucleoside phosphate. The term includes, but is not limited to, a natural nucleotide, a synthetic nucleotide, a modified nucleotide, or a surrogate replacement moiety (e.g., inosine). The nucleoside phosphate may be a nucleoside monophosphate, a nucleoside diphosphate or a nucleoside triphosphate. The sugar moiety in the nucleoside phosphate may be a pentose sugar, such as ribose, and the phosphate esterification site may correspond to the hydroxyl group attached to the C-5 position of the pentose sugar of the nucleoside. A nucleotide may be, but is not limited to, a deoxyribonucleoside triphosphate (dNTP) or a ribonucleo side triphosphate (NTP). The nucleotides may be represented using alphabetical letters (letter designation), as shown in Table 1. For example, A denotes adenosine (i.e., a nucleotide containing the nucleobase, adenine), C denotes cytosine, G denotes guanosine, and T denotes thymidine. W denotes either A or T/U, and S denotes either G or C. N represents a random nucleotide (i.e., N may be any of A, C, G, or T/U). A plus (+) sign preceding a letter designation denotes that the nucleotide designated by the letter is a LNA nucleotide. For example, +A represents an adenosine LNA nucleotide, and +N represents a locked random nucleotide (a random LNA nucleotide). A star (*) sign preceding a letter designation denotes that the nucleotide designated by the letter is a phosphorothioate modified nucleotide. For example, *N represents a phosphorothioate modified random nucleotide.

TABLE 1
Letter designations of various nucleotides.

| Symbol Letter | Nucleotide represented by the symbol Letter |
|---|---|
| G | G |
| A | A |
| T | T |
| C | C |
| U | U |
| R | G or A |
| Y | T/U or C |
| M | A or C |
| K | G or T/U |
| S | G or C |
| W | A or T/U |
| H | A or C or T/U |
| B | G or T/U or C |
| V | G or C or A |
| D | G or A or T/U |
| N | G or A or T/U or C |

As used herein, the term “nucleotide analogue” refers to compounds that are structurally similar (analogues) to naturally occurring nucleotides. The nucleotide analogue may have an altered phosphoate backbone, sugar moiety, nucleobase, or combinations thereof. Generally, nucleotide analogues with altered nucleobases confer, among other things, different base pairing and base stacking proprieties. Nucleotide analogues having altered phosphate-sugar backbone (e.g., Peptide Nucleic Acid (PNA), Locked Nucleic Acid (LNA)) often modify, among other things, the chain properties such as secondary structure formation.

As used herein, the term “LNA (Locked Nucleic Acid) nucleotide” refers to a nucleotide analogue, wherein the sugar moiety of the nucleotide comprises a bicyclic furanose unit locked in a ribonucleic acid (RNA)-mimicking sugar conformation. The structural change from a deoxyribonucleotide (or a ribonucleotide) to the LNA nucleotide is limited from a chemical perspective, namely the introduction of an additional linkage between carbon atoms at 2′ position and 4′ position (e.g., 2′-C, 4′-C-oxymethylene linkage. The 2′ and 4′ position of the furanose unit in the LNA nucleotide may be linked by an O-methylene (e.g., oxy-LNA: 2′-O, 4′-C-methylene-β-D-ribofuranosyl nucleotide), a S-methylene (thio-LNA), or a NH-methylene moiety (amino-LNA), and the like. Such linkages restrict the conformational freedom of the furanose ring. LNA oligonucleotides display enhanced hybridization affinity toward complementary single-stranded RNA, and complementary single- or double-stranded DNA. The LNA oligonucleotides may induce A-type (RNA-like) duplex conformations.

As used herein, the term “oligonucleotide” refers to oligomers of nucleotides or derivatives thereof. The term “nucleic acid” as used herein refers to polymers of nucleotides or derivatives thereof. The term “sequence” as used herein refers to a nucleotide sequence of an oligonucleotide or a nucleic acid. Throughout the specification, whenever an oligonucleotide/nucleic acid is represented by a sequence of letters, the nucleotides are in 5′→3′ order from left to right. For example, an oligonucleotide represented by a letter sequence (W)x(N)y(S)z, wherein x=2, y=3 and z=1, represents an oligonucleotide sequence WWNNNS, wherein W is the 5′ terminal nucleotide and S is the 3′ terminal nucleotide. The oligonucleotides/nucleic acids may be a DNA, a RNA, or their analogues (e.g., phosphorothioate analogue). The oligonucleotides or nucleic acids may also include modified bases, and/or backbones (e.g., modified phosphate linkage or modified sugar moiety). Non-limiting examples of synthetic backbones that confer stability and/or other advantages to the nucleic acids may include phosphorothioate linkages, peptide nucleic acid, locked nucleic acid, xylose nucleic acid, or analogues thereof.

As used herein, the term “primer”, or “primer sequence” refers to a short linear oligonucleotide that hybridizes to a target nucleic acid sequence (e.g., a DNA template to be amplified) to prime a nucleic acid synthesis reaction. The primer may be a RNA oligonucleotide, a DNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acid under such hybridization conditions. The upper limit is often determined by the possibility of having a duplex formation in a region other than the pre-determined nucleic acid sequence in the target nucleic acid. Generally, suitable primer lengths are in the range of about 4 to about 40 nucleotides long.

As used herein, the term “primer comprising a randomizing sequence” refers to a mixture of primer sequences, generated by randomizing a nucleotide at any given location in an oligonucleotide sequence in such a way that the given location may consist of any of the possible nucleotides or their analogues (complete randomization).

In one example, the primer can be a “random primer” or a “complete random primer” or a “chimeric random primer”. Thus the random primer is a random mixture of oligonucleotide sequences, consisting of every possible combination of nucleotides within the sequence. For example, a hexamer random primer may be represented by a sequence NNNNNN or (N)6. A hexamer random DNA primer consists of every possible hexamer combinations of 4 DNA nucleotides, A, C, G and T, resulting in a random mixture comprising 46 (4,096) unique hexamer DNA oligonucleotide sequences. Random primers may be effectively used to prime a nucleic acid synthesis reaction when the target nucleic acid's sequence is unknown.

As used herein, “partially constrained primer” refers to a mixture of primer sequences, generated by completely randomizing some of the nucleotides of an oligonucleotide sequence (i.e., the nucleotide may be any of A, T/U, C, G, or their analogues) while restricting the complete randomization of some other nucleotides (i.e., the randomization of nucleotides at certain locations are to a lesser extent than the possible combinations A, T/U, C, G, or their analogues). For example, a partially constrained DNA hexamer primer represented by WNNNNN, represents a mixture of primer sequences wherein the 5′ terminal nucleotide of all the sequences in the mixture is either A or T. Here, the 5′ terminal nucleotide is constrained to two possible combinations (A or T) in contrast to the maximum four possible combinations (A, T, G or C) of a completely random DNA primer (NNNNNN). Suitable primer lengths of a partially constrained primer may be in the range of about 4 nucleotides to about 40 nucleotides. A complete random primer may contain fully randomized sequence, such as, a dodecamer complete random primer may be represented by a sequence NNNNNNNNNNNN or (N)₁₂. A chimeric random primer may contain a randomized sequence in combination with a specific sequence. For example, a dodecamer chimeric random primer may be represented by a sequence NNNNNNN. Four nucleotides at the 5′ end is constrained to two possible combinations (A or T) in contrast to the maximum four possible combinations (A, T, G or C) of a completely random DNA primer at the 3′ end.

As used herein, the term “plasmid” refers to an extra-chromosomal nucleic acid that is separate from a chromosomal nucleic acid. A plasmid DNA may be capable of replicating independently of the chromosomal nucleic acid (chromosomal DNA) in a cell. Plasmid DNA is often circular and double-stranded.

As used herein, the terms “amplification”, “nucleic acid amplification”, or “amplifying” refer to the production of multiple copies of a nucleic acid template, or the production of multiple nucleic acid sequence copies that are complementary to the nucleic acid template.

As used herein, the term “target nucleic acid” refers to a nucleic acid that is desired to be amplified in a nucleic acid amplification reaction. For example, the target nucleic acid comprises a nucleic acid template.

As used herein, the term “DNA polymerase” refers to an enzyme that synthesizes a DNA strand de novo using a nucleic acid strand as a template. DNA polymerase uses an existing DNA or RNA as the template for DNA synthesis and catalyzes the polymerization of deoxyribonucleotides alongside the template strand, which it reads. The newly synthesized DNA strand is complementary to the template strand. DNA polymerase can add free nucleotides only to the 3′-hydroxyl end of the newly forming strand. It synthesizes oligonucleotides via transfer of a nucleoside monophosphate from a deoxyribonucleoside triphosphate (dNTP) to the 3′-hydroxyl group of a growing oligonucleotide chain. This results in elongation of the new strand in a 5′→3′ direction. Since DNA polymerase can only add a nucleotide onto a pre-existing 3′-OH group, to begin a DNA synthesis reaction, the DNA polymerase needs a primer to which it can add the first nucleotide. Suitable primers comprise oligonucleotides of RNA or DNA. The DNA polymerases may be a naturally occurring DNA polymerases or a variant of natural enzyme having the above-mentioned activity. For example, it may include a DNA polymerase having a strand displacement activity, a DNA polymerase lacking 5′→3′ exonuclease activity, a DNA polymerase having a reverse transcriptase activity, or a DNA polymerase having an exonuclease activity.

As used herein, “a strand displacing nucleic acid polymerase” refers to a nucleic acid polymerase that has a strand displacement activity apart from its nucleic acid synthesis activity. That is, a strand displacing nucleic acid polymerase can continue nucleic acid synthesis on the basis of the sequence of a nucleic acid template strand (i.e., reading the template strand) while displacing a complementary strand that had been annealed to the template strand.

As used herein, the term “complementary”, when used to describe a first nucleic acid/oligonucleotide sequence in relation to a second nucleic acid/oligonucleotide sequence, refers to the ability of a polynucleotide or oligonucleotide comprising the first nucleic acid/oligonucleotide sequence to hybridize (e.g., to form a duplex structure) under certain hybridization conditions with an oligonucleotide or polynucleotide comprising the second nucleic acid/oligonucleotide sequence. Hybridization occurs by base pairing of nucleotides (complementary nucleotides). Base pairing of the nucleotides may occur via Watson-Crick base pairing, non-Watson-Crick base pairing, or base pairing formed by non-natural/modified nucleotides.

As used herein the term “high stringent hybridization conditions” refer to conditions that impart a higher stringency to an oligonucleotide hybridization event than the stringency provided by conditions that may be used for nucleic acid amplification reactions. Higher stringency hybridization conditions may be desired to prevent oligonucleotide hybridization events that may contain mismatched bases within the resulting hybridized duplex. For example, a high stringent hybridization condition may be effected in a nucleic acid amplification reaction by increasing the reaction temperature or by decreasing the salt concentration or by including denaturing agents in the buffer such as glycerol or ethylene glycol. Nucleic acid amplification reactions are sometimes carried out at about 75 mM salt concentrations. In contrast, if a nucleic acid amplification reaction is performed at 15 mM salt concentrations, it may offer a high stringent hybridization condition. Highly stringent hybridization conditions may be used in an in-vitro isothermal nucleic acid amplification reaction by increasing the reaction temperature above the typical reaction temperature of 30° C. For example, the isothermal nucleic acid amplification reaction may be performed at about 35° C. to about 45° C.

As used herein, the term “rolling circle amplification (RCA)” refers to a nucleic acid amplification reaction that amplifies a circular nucleic acid template (e.g., single stranded DNA circles) via a rolling circle mechanism. Rolling circle amplification reaction may be initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). Rolling circle amplification typically produces concatamers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatamers. For example, in a double-primed RCA, one primer may be complementary, as in the LRCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. Rolling circle amplification may be performed in vitro under isothermal conditions using a suitable nucleic acid polymerase such as Phi29 DNA polymerase.

As used herein, the term “multiple displacement amplification” (MDA) refers to nucleic acid amplification methods, wherein the amplification comprises annealing a primer to a denatured nucleic acid followed by strand displacement nucleic acid synthesis. As the nucleic acid is displaced by strand displacement, a gradually increasing number of priming events occur, forming a network of hyper-branched nucleic acid structures. MDA is highly useful for whole-genome amplification for generating high-molecular weight DNA with limited sequence bias from a small amount of genomic DNA sample. Strand displacing nucleic acid polymerases such as Phi29 DNA polymerase or large fragment of the Bst DNA polymerase may be used in multiple displacement amplification. MDA is often performed under isothermal reaction conditions, and random primers are used in the reaction for achieving amplification with limited sequence bias.

As used herein the term “reaction mixture” refers to the combination of reagents or reagent solutions, which are used to carry out a chemical analysis or a biological assay.

One or more embodiments are directed at methods and kits for isothermal nucleic acid amplification reactions using a primer comprising a randomized sequence and a specific primer. These amplification methods are more reliable than currently used amplification techniques and so are more suitable for applications such as amplification of rare sequences where target nucleic acids are available in lower amount (e.g., detection of rare mutant sequences within a population of wild-type sequences), or whole genome amplification reactions.

One or more embodiments of the invention comprise an isothermal amplification reaction using a primer comprising a randomized sequence and a specific primer for amplifying a template nucleic acid sequence. One or more embodiments also comprise capturing the amplified nucleic acid sequence using specific primers during the isothermal amplification reaction. The primer comprising a randomized sequence and the specific primer are both present in the same reaction mixture for simultaneous amplification, capture and the subsequent amplification of the captured nucleic acid sequences. The specific primer may be attached to a substrate. At one example of the methods may be used to generate a substrate coated with nucleic acid.

In one or more of the embodiments, the primer comprising a randomized sequence may comprise at least one modified nucleic acid base. Such primers, when used, typically require high salt or low temperature conditions for efficient hybridization to the template nucleic acid sequence to initiate amplification reaction. The modified nucleic acid base present in the primer, used in one or more of the methods, is capable of increasing the melting temperature (Tₘ) of the primer (with randomized sequence).

In some embodiments, the primer comprising a randomized sequence is a partially constrained primer. Suitable lengths of the partially constrained primer may be in the range of about 4 to about 10 nucleotides. A combination of partially constrained primers having varying primer lengths may also be used. The primer comprising a randomized sequence (e.g., partially constrained primer) may comprise modified nucleic acid bases, which increased Tₘ of the primer. The amplification by random primer and specific primer, and subsequent capture by specific primer in the same reaction mixture under the same conditions, may be used to further increase the efficiency of the amplification.

The nucleic acid is amplified by contacting the nucleic acid template with a DNA polymerase and deoxyribonucleoside triphosphate and incubating the reaction mixture under conditions suitable for nucleic acid amplification. The amplification of the nucleic acid template may be performed under isothermal conditions. In some embodiments, the nucleic acid template is amplified using isothermal nucleic acid amplification by RCA methods.

In one or more embodiments, the primer comprising a randomized sequence may comprise a completely random DNA primer (NNNNNN). In one or more other embodiments, the primer may comprise a partially constrained primer, wherein some of the nucleotides of an oligonucleotide sequence are randomized (WWWNNN). In one or more embodiments, the primer may comprise a specific sequence at the 5′ end and a random sequence at the 3′ end.

As noted, suitable lengths of the random primer may be in the range of 4 nucleotides to 10 nucleotides long. In some embodiments, the length of the random primer is 5 to 6 nucleotides. In some embodiments, comprising a partially constrained primer, the primer is about 5 to about 7 nucleotides long. One potential disadvantage of short random primers is that short primers with a randomized sequence have low melting temperatures. By introducing modified nucleic acid bases to the short random primer, the melting temperature of the primer may be increased. Suitable modified nucleic acid bases include, but is not limited to, may be a locked nucleic acid base, a peptide nucleic acid base or a ribonucleic acid base.

In some embodiments, the primer comprising a randomized sequence may be a partially constrained primer. The partially constrained primer comprises, at suitable locations, nucleic acid analogues that have higher complementary specificity than that of natural nucleotides (e.g., Locked Nucleic Acid (LNA) nucleotides). The location of nucleotide analogues in the partially constrained primer may be chosen in such a way that it hybridizes specifically to a complementary sequence present in the template nucleic acid sequence under nucleic acid amplification reaction conditions. When the partially constrained primer comprising LNA nucleotide is used for nucleic acid amplification reaction, the amplification reaction may be performed at more stringent hybridization conditions. The more stringent conditions may be beneficial for the DNA polymerase. The amplification reaction may be performed at higher temperatures (e.g., above 30° C. for an isothermal nucleic acid amplification), the upper limit being the temperature at which the DNA polymerase used in the reaction may become non-functional. It may also be performed at a lower salt concentration (e.g., about 10 μM to about 25 μM salt concentration) than what is normally used (e.g., about 75 μM salt concentration). Due to higher complementary specificity, the hybridization of the partially constrained primer comprising LNA nucleotides to the target nucleic acid may not be substantially affected by high stringent hybridization conditions. Hence, the amplification of the desired target nucleic acid amplification may also not be substantially affected.

The stringent condition for hybridization is significant, as short random primers typically require high salt or low temperature condition for efficient hybridization to initiate DNA amplifications. However the said condition is not suitable for the single stranded amplified product DNA to hybridize to the specific primer correctly. The specific primers generally are desired to be longer, and thus require high stringency hybridization conditions to provide correct specificity for hybridization. In this case, the use of constrained random primers along with modified nucleotides may be a solution to have amplifications at high stringency conditions. Since the Tₘ of the constrained random primers are high, even at high stringency conditions, the primers with randomized sequence are able to hybridize to the template DNA to make the amplification reaction.

The partially constrained primers may be generated by completely randomizing (i.e., the nucleotide base may be any of A, T/U, C, G or their analogues) one or more nucleotides of an oligonucleotide sequence, while restricting the complete randomization of some other nucleotides (i.e., the randomization of nucleotide bases at certain locations are to a lesser extent than the four possible combinations A, T/U, C or G). In some embodiments, randomization of two nucleotides in the partially constrained primer is restricted. In some embodiments, the randomization of more than two nucleotides (e.g., three, four, or five nucleotides) in the partially constrained primer is restricted. The extent of randomization may be empirically determined based on amplification reaction requirements and reaction conditions.

In some embodiments, the partially constrained primer may comprise a nucleotide analogue at a suitable position. In some embodiments, a nucleotide analogue, that has higher complementary specificity than that of a natural nucleotide, may be used. Non-limiting examples of suitable nucleic acid analogues that may be incorporated in the partially constrained primer include peptide nucleic acids (PNA), 2′-fluoro N3-P5′-phosphoramidates, 1′,5′-anhydrohexitol nucleic acids (HNA), ribonucleic acid (RNA) or locked nucleic acid (LNA) nucleotides. Due to higher complementary specificity of the nucleotide analogues, a nucleic acid amplification reaction using partially constrained primers comprising nucleotide analogues may be performed at more stringent conditions (e.g. performing the reaction at higher temperatures or lower salt concentration). The partially constrained primer having nucleotide analogues has higher complementary specificity to the target (for example, the Tₘ of the target nucleic acid-primer complex may be higher when the partially constrained primer comprises the nucleotide analogue). Since such primers hybridizes to the target nucleic acid even at higher temperatures/lower salt concentration, the desired target nucleic acid amplification is not substantially affected under stringent hybridization conditions.

In some embodiments, the partially constrained primer comprises an LNA nucleotide at a suitable position. Suitable LNA nucleotides include, but are not limited to, an oxy-LNA (2′-O, 4′-C-methylene-β-D-ribofuranosyl nucleotide), a thio-LNA (2′-S, 4′-C-methylene-β-D-ribofuranosyl nucleotide), or an amino-LNA (2′—NH, 4′-C-methylene-β-D-ribofuranosyl nucleotide) nucleotide. LNA nucleotides may be located toward the 5′ end of the partially constrained primer sequence. In some embodiments, the partially constrained primer comprises two LNA nucleotides. For example, a partially constrained primer may have a LNA nucleotide at the 5′ terminal position, and also at the position adjacent to the 5′ terminal positions. In other examples, the 5′ terminal nucleotide of the partially constrained primer may be a natural nucleotide whereas the next two nucleotides adjacent to the 5′ terminal nucleotide may be LNA nucleotides. Polymerase efficiency is better when the LNA nucleotide is located in a region, which is greater than 1 or 2 bases from the 3′ end of the primer, than the case where the LNA nucleotide is located within 1 or 2 bases from the 3′ end of the primer (the ultimate or penultimate base).

In one or more embodiments, the specific primers present in the reaction mixture also take part in the reactions. The specific primer used in the amplification reaction is long primer, such as, for example, 10 to 20 nucleotide sequences or 15 to 20 nucleotide sequences. The melting temperature of long specific primers is generally high and they may hybridize, in some embodiments, only in more stringent conditions such as, low salt and high temperature conditions. The sequence of adding the primers to the reaction mixture is not significant, because the amplification reaction initiates in the presence of both specific primers and primers comprising a randomized sequence. One advantage of having both primers in the same reaction is the simultaneous amplification of various loci present in the template nucleic acid sequence. In some embodiments, the specific primers may have a high specificity for a particular locus, for example, for locus 2 in between 5 loci that may be present in the template. Simultaneous amplification, by a primer comprising a randomized sequence and by a specific primer, may result in an amplified nucleic acid sequence with all 5 loci (by primer comprises randomized sequence) and also the amplified nucleic acid sequence with only locus 2 (by specific primer). In some embodiments, the primer comprising a randomized sequence may not be able to efficiently support amplification of some of the loci, which can be amplified by specific primer. For example, the primer comprising a randomized sequence is not able to amplify loci 2, 3 and 4; however the specific primer is able to amplify these loci, which results in a population of amplified nucleic acid sequences comprising loci 1 and 5 along with high expression of loci 2, 3, and 4. In such embodiments, the rate of missing a particular locus present in the template nucleic acid sequence is decreased. One or more of the embodiments also increase the rate of amplification and expression of various loci by using both random and specific primer.

One or more of the examples of the methods, comprise providing a plurality of specific primers, wherein the plurality of specific primers comprise a first specific primer, a second specific primer, a third specific primer and a fourth specific primer. In one embodiment, a template DNA comprises a plurality of loci, such as loci 1, 2, 3 and 4. The first, second, third and fourth specific primers hybridize to the loci 1, 2, 3 and 4 respectively present on the template DNA and amplifying the loci 1, 2, 3 and 4 to form first, second, third and the fourth amplified nucleic acid sequences. Therefore, the use of random primer decreases the possibility of under-amplification of each locus during amplification.

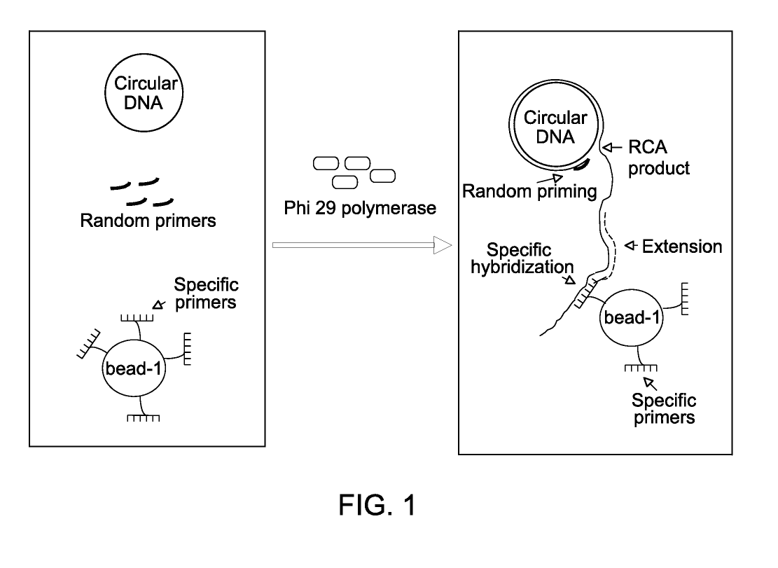

One example of the method of amplification using both random primer and substrate-bound (e.g. bead-1-bound) specific primer is illustrated in FIG. 1. The amplification reaction represents rolling circle amplification using a circular DNA as a template and a random primer in the presence of Phi 29 DNA polymerase. The bead-1-bound specific primer hybridizes to the amplified DNA and subsequently extends from the specific primer end.

Strand displaced single stranded DNA is created by the random primed amplification of template DNA using phi29 DNA polymerase. Single stranded DNA can be hybridized to an additional primer, which is a specific primer present in the reaction wherein the primer is attached to a substrate (as shown schematically in FIG. 1). The method comprises capturing of the amplified nucleic acid sequence by hybridization to the specific primer attached to a first substrate (bead-1, as described in FIG. 1) to form a first substrate-bound nucleic acid sequence. The method further comprises extension of a nucleic acid sequence from the hybridization site of the specific primer using the first substrate-bound (bead-1-bound) nucleic acid sequence as a template.

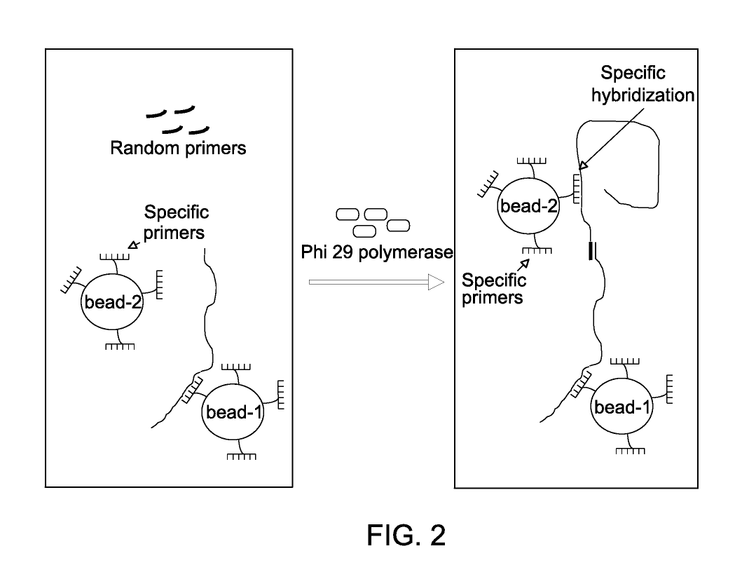

One more example of the method further comprises amplifying the first substrate-bound nucleic acid sequence (bead-1 bound nucleic acid as shown in FIG. 2) by a primer comprising a randomized sequence. The amplification results a second amplified nucleic acid sequence. The method further comprises capturing the second amplified nucleic acid sequence by a second substrate (bead-2, as shown in FIG. 2) by hybridization of the second amplified nucleic acid with a specific primer attached to the second substrate (bead-2) (as shown schematically in FIG. 2). The amplified nucleic acid sequence, as referred to herein as a ‘first amplified nucleic acid sequence’, is attached to a first substrate-bound (bead-1-bound) specific primer and may be further amplified by a random primer to form a ‘second amplified nucleic acid sequence’ which is captured by a second substrate (bead-2) bound specific primer, as schematically represented in FIG. 2. The second amplified nucleic acid sequence may be further amplified by a second-substrate bound specific primer after hybridization, to form a third amplified nucleic acid sequence. Therefore, the first substrate-bound single stranded nucleic acid sequence is amplified by a random primer and captured by a specific primer attached to a second substrate, and transferred to the second substrate.

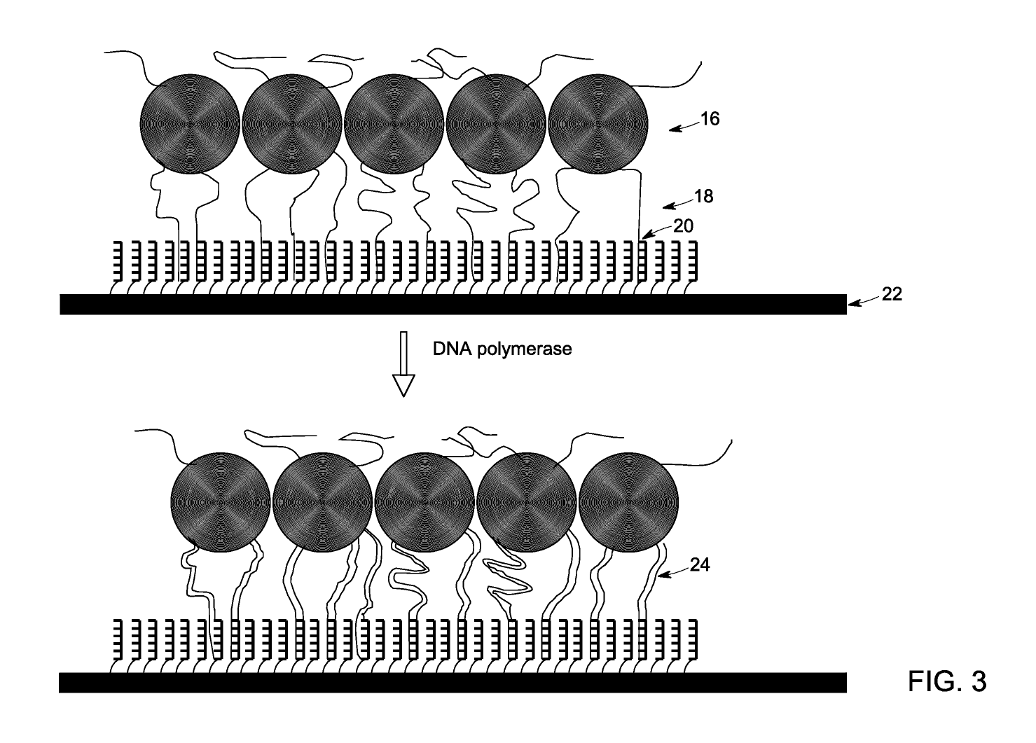

In one example, after capturing the first-substrate (such as a bead) (16) bound amplified nucleic acid sequence (18) by a second-substrate (22) bound specific primer (20), there is an extension of nucleic acid sequence from said primer end results a double stranded nucleic acid sequence (24) bound to the second substrate, which is schematically illustrated in FIG. 3.

The amplification reaction is isothermal, unlike temperature cycling reactions. Non-limiting examples of suitable isothermal nucleic acid amplification reactions that may be used comprise, but are not limited to, rolling circle amplification (RCA) or multiple displacement amplification (MDA). The methods may be used, for example, in the amplification of circular nucleic acid templates or linear nucleic acid templates. The methods may be effectively used even when the amount of the nucleic acid template to be amplified is minimal. The methods may be useful, for example, in whole-genome amplification or in single nucleic acid amplification reactions.

Non-limiting examples of isothermal nucleic acid amplification methods include LCR, self-sustained sequence replication (SSR), NASBA, LAMP, amplification with Qb-replicase, or the like. In some embodiments, the nucleic acid template is amplified using SDA. In some embodiments, the nucleic acid template is amplified using MDA. In one embodiment, the nucleic acid template is amplified using RCA method. RCA could be used as a LRCA or it may be an ERCA. In another embodiment, MPRCA is employed for amplifying the nucleic acid template.

The nucleic acid polymerase that is used for amplification may be a proofreading or a non-proofreading nucleic acid polymerase. In some embodiments, the nucleic acid polymerase used is a strand displacing nucleic acid polymerase. The nucleic acid polymerase may be a thermophilic or a mesophilic nucleic acid polymerase. Examples of DNA polymerases that are suitable for use include, but are not limited to, Phi29 DNA polymerase, hi-fidelity fusion DNA polymerase (e.g., Pyrococcus-like enzyme with a processivity-enhancing domain, New England Biolabs, MA), Pfu DNA polymerase from Pyrococcus furiosus (Strategene, Lajolla, Calif.), Bst DNA polymerase from Bacillus stearothermophilus (New England Biolabs, MA), Sequenase™ variant of T7 DNA polymerase, exo(−) Vent_{R}™ DNA polymerase (New England Biolabs, MA), Klenow fragment from DNA polymerase I of E. coli, T7 DNA polymerase, T4 DNA polymerase, DNA polymerase from Pyrococcus species GB-D (New England Biolabs, MA), or DNA polymerase from Thermococcus litoralis (New England Biolabs, MA).

In some embodiments, the methods may employ a highly processive, strand-displacing polymerase to amplify the nucleic acid template under conditions for high fidelity base incorporation. A high fidelity DNA polymerase refers to a DNA polymerase that, under suitable conditions, has an error incorporation rate equal to or lower than those associated with commonly used thermostable PCR polymerases such as Vent DNA polymerase or T7 DNA polymerase (from about 1.5×10⁻⁵ to about 5.7×10⁻⁵). Additional enzymes may be included in the amplification reaction mixture to minimize mis-incorporation events. For example, protein mediated error correction enzymes, such as, MutS, may be added to improve the polymerase fidelity either during or following the polymerase reaction.

In some embodiments, the amplification reaction employs a DNA polymerase that generates single stranded, amplified DNA after amplification. The DNA polymerase is capable of strand displacement DNA synthesis. The polymerase is capable of creating a single stranded DNA followed by synthesizing a new strand to form a double stranded DNA. In one embodiment, once the primer bound to the template nucleic acid, the DNA polymerase initiates nucleic acid polymerization in 3′ to 5′ direction, at the same time displacing any blocking strand by displacing it in a 5′ to 3′ direction.

In some embodiments, a Phi29 DNA polymerase or Phi29-like polymerase may be used for amplifying a DNA template. In some embodiments, a combination of a Phi29 DNA polymerase and another DNA polymerase may be used.

The nucleic acid template may be a single-stranded nucleic acid template or it may be a double-stranded nucleic acid template. It may be a circular nucleic acid template, a nicked nucleic acid template, or a linear nucleic acid template. The nucleic acid template may comprise DNA and/or RNA, or a DNA-RNA chimeric template. In some embodiments, the nucleic acid template may be a DNA template. The DNA template may be a cDNA or a genomic DNA. The circular nucleic acid template may be a synthetic template (e.g., a linear or nicked DNA circularized by enzymatic/chemical reactions), or it may be a plasmid DNA. The nucleic acid template may be a synthetic nucleic acid or a natural nucleic acid. It may also comprise modified nucleotides. In one example embodiment, the nucleic acid template is a circular DNA template.

The template DNA may, for example, be collected from a patient or a donor. In one example, template DNA are collected from a patient, followed by amplification of the DNA, and then captured on a substrate for sequencing and analysis. The amplified template DNA is used for detection of specific locus, single nucleotide polymorphism (SNP), or restriction fragment length polymorphism (RFLP). The template DNA may be recovered, for example, from hair roots, red blood cells, epithelial cells, saliva or pathological specimens and the amplified DNA may be subjected to forensic analysis or molecular diagnostics.

The nucleic acid template may comprise a recombination site. The recombination site comprises nucleic acid sequences that are favorable for recombination. In one embodiment, a nucleic acid template may be engineered to comprise a recombination site, and amplifying the engineered nucleic acid template generates the amplified nucleic acid comprising the recombination sites. Engineering of the nucleic acid template may be achieved by any of the genetic engineering or molecular biology techniques known in the art, such as, but not limited to, cloning. In some embodiments, the recombination site may be a site-specific recombination site. The site-specific recombination site refers to a recombination site comprising specific sequences, which is recognized by a specific recombination protein.

The nucleic acid template may be amplified to generate an amplified nucleic acid in a solution, suitable for performing a nucleic acid amplification reaction. In some embodiments, a circular DNA template may be amplified by rolling circle amplification. In some other embodiments, a linear DNA template may be amplified using multiple displacement nucleic acid amplification.

Each of the reagents used in the nucleic acid amplification reaction may be pre-treated to remove any contaminating nucleic acid sequences. The pre-treatment of the reagents may include, but is not limited to, incubating the reagents in the presence of ultraviolet radiation. The reagents may also be decontaminated, for example, by incubating the reagents in the presence of a nuclease and its cofactor (e.g., a metal ion). Suitable nucleases include, but are not limited to, exonucleases such as exonuclease I or exonuclease III. Proofreading DNA polymerases that may be used in a DNA amplification reaction may be decontaminated, for example, by incubating with a divalent metal ion (e.g., magnesium or manganese). The free nucleotides employed in nucleic acid template amplification may include, but are not limited to, natural nucleotides (e.g., dATP, dGTP, dCTP, or dTTP) or their modified analogues. Other components such as buffers, salts and the like may also be added.

Upon DNA template amplification, the amplified DNA may be captured by employing a substrate-bound specific primer that is homologous to at least some part of the amplified DNA. The substrate-bound specific primers may capture the amplified nucleic acid sequence, for example, by hybridization generating substrate-bound amplified DNA. Additionally, in the same reaction, the substrate-bound primer may be further extended by the DNA polymerase, in a subsequent DNA amplification reaction to create additional amounts of DNA captured on the substrate.

In some embodiments, the specific primer may be attached to a substrate or a capturing agent. The substrate can be a first substrate, a second substrate, a third substrate and so on. The substrate may be, for example, a bead. The material of the substrate may be, for example, selected from polymer, glass, or metal. In one embodiment, the material of the substrate is polymer. The capturing agent may be an affinity tag. The specific primer may be attached to a substrate or a capturing agent by various interactions. For example, the specific primer may be attached to the capturing agent via nucleic acid hybridization, covalent linkage, electrostatic interaction, Van der Waals interactions, hydrophobic interaction, or a combination of these. For example, the specific primer may be covalently attached to a substrate made of polymer. Upon DNA amplification reaction, the amplified DNA may be captured, for example, by a substrate made of polymer. The amplification reaction may comprise different specific primers, wherein each of them may be attached to a different type of capture agent. A series of specific primers may also be attached to a first substrate, a second substrate, a third substrate, and so on. The first, or second, or third substrate may comprise, for example, beads, test tubes, multi-well plates, slides and eppendorfs.

As noted, the amplified copies of the nucleic acid template may be attached to a capture bead. As non-limiting examples, these attachments may be mediated by chemical groups or oligonucleotides that are bound to the surface of the bead. The amplified copies of the nucleic acid template may be attached to a solid support such as, but not limited to, a capture bead or other suitable surfaces in any suitable manner known in the art. For example, the amplification copies of the nucleic acid template may be attached to the substrate-bound specific primer by hybridization.

The specific primer may be attached to a substrate (or a first substrate) or a capturing agent either directly or via a linker. The specific primer attached to a substrate via a linker may be used for purification of nucleic acid having a complementary sequence to the specific primer. The first substrate is selected from a bead or a surface. The capturing agent is selected from an affinity tag, or a polymer. In one nonlimiting embodiment, the linker may be a polymer, such as acrylamide, dextran, or poly ethylene glycol (PEG). In at least one embodiment, one end of a linker may comprise a reactive group (such as an amide group), which forms a covalent bond with the specific primer to be immobilized. The specific primer may be bound to the DNA capturing agent, such as an affinity tag, by covalent linkages, such as chelation. The affinity tag may comprise, but is not limited to, histidine (His-tag), or biotin.

For example, the amplified nucleic acid may be captured by a specific primer attached to an affinity tag to form affinity tag-bound amplified nucleic acid sequence. The affinity tag-bound amplified nucleic acid sequence may then be subsequently captured by a suitable substrate for a specific tag. For example, the amplified nucleic acid captured by a biotinylated specific primer may further be captured by a streptavidin bead by formation of a biotin-streptavidin complex. Affinity tags may be selected so that they may be captured using methods that do not involve nucleic acid hybridization. For example, affinity tags may be captured by covalent linkage, electrostatic interaction, Van der Waals interactions, hydrophobic interaction, or a combination of these. The affinity tag may be used, for example, to purify different species of amplified nucleic acids from the amplification reaction.

The beads may be of any suitable size and may be fabricated from materials selected from, but not limited to, inorganics, natural polymers, or synthetic polymers. Specific examples of these materials include, but not limited to, cellulose, cellulose derivatives, acrylic resins, glass, silica-gels, polystyrene, gelatin, polyvinyl pyrrolidone, co-polymers of vinyl and acryl amide, polystyrene cross-linked with divinylbenzene, dextran, polyacrylamide, cross-linked dextran (e.g., Sephadex™) agarose gel (Sepharose™) or other solid phase supports known in the art. For example, the capture beads may have a diameter of about 1 to 400 μm.

In one or more embodiments, covalent chemical attachment of a specific primers sequence to the bead may be accomplished by using standard coupling agents. For example, water-soluble carbodiimide may be used to link the 5′-phosphate of a specific primers sequence to amine-coated capture beads through a phosphoamidate bond. Other linkage chemistries, that may be used to join the oligonucleotide to the beads, include, but are not limited to, N-hydroxysuccinamide (NHS) and its derivatives.

In one or more embodiments, the capture agent, such as capture bead, may be designed to have a plurality of specific primers that recognize or complement a portion of the nucleic acid template, and the amplification copies of this template. For example, to obtain clonal-amplification of the template, one unique nucleic acid species may be used to attach to any one capture bead. One or more of the amplification methods may be used to generate DNA-coated beads, which, for example, may be used to mimic plasmids found in bacterial colonies. The methods may also, for example, be used to generate an array of different DNA sequences that can be used for downstream purposes such as DNA sequencing or DNA detection.

In one embodiment, amplification may be performed in an emulsion. The template may be captured to the bead prior to emulsification or after the emulsion has been formed. In another embodiment, the surface-bound specific primer may be located on beads in an emulsion droplet to allow production of different DNA-coated beads. For example, an emulsion may be created in which each droplet may contain a single DNA molecule of interest, either alone or in addition to other DNA molecule. If each bead is present in each emulsion droplet, the bead with amplified product DNA may be subsequently washed after capturing to remove unbound DNA. The washed beads may then be used for additional amplification reactions. The washed beads with the amplified nucleic acid may be used to create a “DNA library” if individual DNA molecules have been initially segregated into the emulsion droplets. Individual bead from the population can be isolated to create “DNA clones” in solution by subsequent DNA amplification of the bead-bound DNA. The bead-bound amplified DNA may be used for many different types of analysis (e.g., protein expression or cloning). The beads may also be used to create a “DNA array”. By creating a monolayer of the beads, which may be attached to the surface, a non-overlapping randomized bead array may form in which each bead is attached to the product DNA.

The beads in emulsion may also be used as templates for additional DNA amplification by strand displacement synthesis and capture the product by hybridization. The emulsion may be generated after adding beads to an amplification solution. The capturing agent, such as capture beads, with or without attached nucleic acid template may be suspended in a heat-stable oil-in-water emulsion. There may be microdroplets with bead but without any nucleic acid, or with nucleic acids but without any bead, or without any nucleic acids or without any bead. There may be microdroplets with more than one copy of nucleic acid. The emulsion or micro droplet may be formed by any suitable methods including, but not limited to, adjuvant methods, counter-flow methods, cross current methods, rotating drum methods, and membrane methods.

The beads in emulsion may be treated under chemical or thermal denaturation conditions to yield beads with single stranded DNA. The single stranded DNA is created by extension of the attached primers hybridized to a single stranded product during the amplification reaction. The beads with the amplified nucleic acid may be used for downstream methods that require single stranded DNA such as, sequencing by hybridization or sequencing by ligation or sequencing by synthesis.

In some embodiments of the kit for amplifying nucleic acid, the kit comprises a Phi29 DNA polymerase, a primer comprising a randomized sequence, and a specific primer. The specific primer may be attached to a substrate or a capture agent. The kits may further comprise other reagents, known in the art that are useful in nucleic acid amplifications. The kit may also comprise a nucleic acid polymerase and a partially constrained primer. The nucleic acid polymerase and the partially constrained primer may be packaged in a single vessel or they may be packaged in separate vessels.

In one embodiment, the kit comprises a Phi29 DNA polymerase and a partially constrained primer. The partially constrained primer in the kit may comprise a nucleotide analogue, such as a LNA nucleotide. In some embodiments, the partially constrained primer is a DNA-LNA chimera primer. The partially constrained primer in the kit may be a nuclease-resistant primer, for example, an exonuclease-resistant primer. These exonuclease-resistant primers in the kit may contain one or more phosphorothioate linkages between the nucleotides.

The kit may further comprise reagents or reagent solutions required for performing a nucleic acid amplification reaction. It may further include an instruction manual detailing the specific components included in the kit, or the methods for using them in nucleic acid amplification reactions, or both.

### Example 1

### Decontamination of Reaction Mixture Before Amplification

The reagents and reagent solutions that were used for nucleic acid amplification reaction were decontaminated in a nucleic acid-free hood prior to their use to remove any contaminating nucleic acids. The reagents such as Phi29 DNA polymerase, exonuclease I, exonuclease III, and SSB protein were stored in 50 mM Tris-HCl (pH 7.2), 200 mM NaCl, 10 mM DTT, 1 mM EDTA, 0.01% (v/v) Tween-20, and 50% (v/v) glycerol. The primer-nucleotide solution (primer-nucleotide mix) comprising primer and nucleotides (dNTPs) was decontaminated by incubating the primer-nucleotide mix with a combination of exonuclease I, exonuclease III, and a single stranded DNA binding protein (SSB protein). The enzyme mix comprising a DNA polymerase was decontaminated by incubating with an exonuclease in presence of a divalent cation (e.g., Mg²⁺). Any target nucleic acid amplification reaction was performed using the decontaminated enzyme mix and the primer-nucleotide mix.

As shown in Table 2, the enzyme mix containing 200 ng of Phi29 DNA polymerase was incubated with 0.1 unit of exonuclease III in 50 mM HEPES buffer (pH=8.0) containing 15 mM KCl, 20 mM MgCl₂, 0.01% (v/v) Tween-20, and 1 mM tris (2-carboxyethyl)phosphine (TCEP). The incubation was performed either at 30° C. for about 60 min., or at 4° C. for 12 h. The incubated enzyme mix was then transferred to an ice-bath, and was used in DNA amplification reactions as such without any inactivation of the exonuclease III. This small amount of exonuclease III had no substantial effect on the amplification reaction if the finished amplification reaction was treated immediately upon completion to inactivate the exonuclease.

To decontaminate the primer-nucleotide mix, it was incubated with a combination of exonuclease I, exonuclease III and SSB protein as shown in Table 2. The incubation was performed at 37° C. for about 60 min in 50 mM HEPES buffer (pH=8.0) containing 15 mM KCl, 20 mM MgCl₂, 0.01% (v/v) Tween-20 and 1 mM TCEP (Total reaction volume was 5 μL). E. coli SSB protein was used in this example as a suitable single-stranded binding protein. After decontamination of the primer-nucleotide mix, the exonucleases were thermally inactivated by incubating the primer-nucleotide mix at 85° C. for about 15 min., followed by incubation at 95° C. for about 5 min to about 10 min.

TABLE 2
Decontamination of the enzyme mix and primer-nucleotide mix solutions.

| | Primer- | DNA polymerase |
|---|---|---|
| | nucleotide mix | (enzyme) mix |
| | (each reaction) | (each reaction) |
| 2× Reaction buffer (reaction buffer | 2.6 μL | 2.5 μL |
| is 50 mM HEPES buffer | | |
| (pH = 8.0), 15 mM KCl, 20 mM | | |
| MgCl2, 0.01% Tween-20 and 1 mM | | |
| TCEP) | | |
| Distilled water | — | 2.2 μL |
| 10 mM dNTP mix | 0.4 μL | — |
| 1 mM primer | 0.4 μL | — |
| Exonuclease I (20 unit/μL) | 0.5 μL | — |
| Exonuclease III (10 unit/μL) | 0.1 μL | — |
| Exonuclease III (1 unit/μL) | — | 0.1 μL |
| SSB protein (100 ng/μL) | 1 μL | — |
| Phi29 DNA polymerase (1 mg/ml) | — | 0.2 μL |
| Total reaction volume | 5 μL | 5 μL |

### Example 2

### No Template Control Amplification

Non-specific amplification reaction during a nucleic acid amplification reaction was estimated by performing a DNA amplification reaction without any added template DNA (No Template Control (NTC) amplification). The reactions employed either a completely random primer, or a partially constrained primer that comprises LNA nucleotides. Both these primers were exonuclease-resistant primers, having phosphorothioate linkages between the nucleotides toward the 3′ end of the sequence.

The amplification products from a DNA amplification reaction with no added target DNA template (NTC) arise from non-specific amplification reactions (false amplification or background amplification). The non-specific amplification may be due to amplification of contaminating DNA molecules, or background DNA molecules captured by beads by bead-bound specific primer. To avoid any non-specific amplification reaction originating from contaminating DNA, all the reagents or reagent solutions (enzyme mix and primer-nucleotide) that were used for the amplification reaction were decontaminated to remove any contaminating DNA using the procedure described in Example 1.

For estimating non-specific DNA amplification reactions, DNA amplification reaction was performed by incubating the decontaminated primer-nucleotide mix and the decontaminated enzyme mix at 30° C. for about 400 min without any added DNA template. The amplification reaction mixture was composed of 40 μM primer (random primer, or partially constrained primer of sequence, W+W+NN*S in which W denotes either A or T/U, and S denotes either G or C, N represents a random nucleotide (i.e., N may be any of A, C, G, or T/U), a plus (+) sign preceding a letter designation denotes that the nucleotide designated by the letter is a LNA nucleotide, a star (*) sign preceding a letter denotes that the nucleotide designated by the letter is a phosphorothioate modified nucleotide.); 400 μM dNTPs (equal mixture of each of dATP, dCTP, dGTP, dTTP); and 200 ng phi29 DNA polymerase (200 ng per 10 μL reaction). The reaction mixture was incubated in 50 mM HEPES buffer (pH=8.0) containing 15 mM KCl, 20 mM MgCl₂, 0.01% (v/v) Tween-20, 1 mM TCEP.

### Example 3

### Amplification Reaction

For nucleic acid amplification reaction, the primer-nucleotide mix and the enzyme mix were mixed together after decontamination along with template nucleic acid to create an amplification reaction, which was then incubated at about 30° C. The isothermal amplification reaction was performed in presence of Phi 29 DNA polymerase in presence of random primer, bead-bound specific primer, and pUC18 plasmid DNA.

For estimating DNA amplification reactions, DNA amplification reactions were performed by incubating the de-contaminated primer-nucleotide mix and the de-contaminated enzyme mix at 30° C. for about 400 min with pUC18 plasmid DNA template. The amplification reaction mixture was composed of 40 μM primer (random primer, or partially constrained primer of sequence W+WNN*S in which W denotes either A or T/U, and S denotes either G or C, N represents a random nucleotide (i.e., N may be any of A, C, G, or T/U) a plus (+) sign preceding a letter designation denotes that the nucleotide designated by the letter is a LNA nucleotide, a star (*) sign preceding a letter denotes that the nucleotide designated by the letter is a phosphorothioate modified nucleotide.);); 1 μL specific primer-conjugated beads (approximately 70,000 beads per microliter, wherein specific primer is covalently attached to the beads); 400 μM dNTPs (400 μM each of dATP, dCTP, dGTP, dTTP); 1 pg pUC 18 plasmid DNA, and 200 ng phi29 DNA polymerase (200 ng per 10 μL reaction). The reaction mixture was incubated in 50 mM HEPES buffer (pH=8.0) containing 15 mM KCl, 20 mM MgCl₂, 0.01% (v/v) Tween-20, 1 mM TCEP.

### Example 4

### Capture-Bead Synthesis

DNA capture bead, in which a specific primer is attached to the bead, is synthesized as follows. N-hydroxysuccinimide ester (NHS)-activated Sepharose (NHS HP SpinTrap™, GE Healthcare, Piscataway, N.J.) beads were used for synthesizing DNA capture beads. The beads were then functionalized with oligonucleotide using protocols described in the product literature (GE Healthcare NHS HP SpinTrap™ Protocol). Amine-labeled, HEG (hexaethyleneglycol) linker attached to the 5′ end of the −40 universal capture primer which is complementary to a section of one strand of (the template to be amplified) pUC 18 plasmid DNA (5′-Amine-3 HEG spacers/5AmMC6/GTTTTCCCAGTCACGACGTTG*T*A-3′; SEQ ID NO:1) was commercially obtained from IDT Technologies (Coralville, Iowa, USA) where 5AmMC6 indicates that a primary amine group attached by a hexaethylene glycol linker to the 5′ end of the oligonucleotide. The capture primers were dissolved in TE buffer, pH 8.0, to obtain a final concentration of 1 mM. 3 pmoles of primer were bound to each 1 μL beads and the packed slurry of 1 μL primer-conjugated beads contains between 50,000 and 70,000 individual beads wherein a bead comprises a diameter of approximately 30μ meter. This would result in approximately 3 pmoles of primer attached to 60,000 beads, or 1.5×10̂8 primers per bead if the attachment reaction went to 100% completion.

### Example 5

### Capture of Amplified DNA on Beads

Capture bead comprises specific primer sequence or sequences attached to it. The DNA capture beads (Bead-Spacer-Seq. ID No. 1 or Bead-Spacer-Seq. ID No. 2) were utilized to capture amplified DNA molecules as follows. A DNA amplification reaction was performed by incubating the de-contaminated primer-nucleotide mix and the de-contaminated enzyme mix at 30° C. for about 400 min with pUC18 plasmid DNA template on a rotator platform to insure beads remain in suspension during the reaction. The amplification reaction mixture was composed of 40 μM primer (partially constrained primer of sequence W+WNN*S in which W denotes either A or T/U, and S denotes either G or C, N represents a random nucleotide (i.e., N may be any of A, C, G, or T/U) a plus (+) sign preceding a letter designation denotes that the nucleotide designated by the letter is a LNA nucleotide, a star (*) sign preceding a letter denotes that the nucleotide designated by the letter is a phosphorothioate modified nucleotide.); 3 μL specific primer (approximately 70,000 beads per microliter, specific primer is covalently attached to the beads); 400 μM dNTPs (400 μM each of dATP, dCTP, dGTP, dTTP); 2 ng pUC18 plasmid DNA, and 200 ng phi29 DNA polymerase (200 ng per 10 μL reaction). The incubation was performed in 50 mM HEPES buffer (pH=8.0) containing 15 mM KCl, 20 mM MgCl₂, 0.01% (v/v) Tween-20, 1 mM TCEP. After the reaction the beads were allowed to settle. The reaction supernate, containing unbound DNA amplification product was removed from the reaction tube and reserved for later analysis. The remaining bead pellet was then washed with 200 μL of TE by addition of the TE, brief agitation by vortexing, and brief centrifugation using a microfuge. The TE supernate from this step was removed and the washing step repeated by an additional two times. The washed pellet was suspended in 97 μL TE. 15 μl of TE-washed capture beads from the amplification reaction as described above was transferred to a petri dish. The bead-slurry was allowed to settle and individual bead was isolated by micromanipulation.

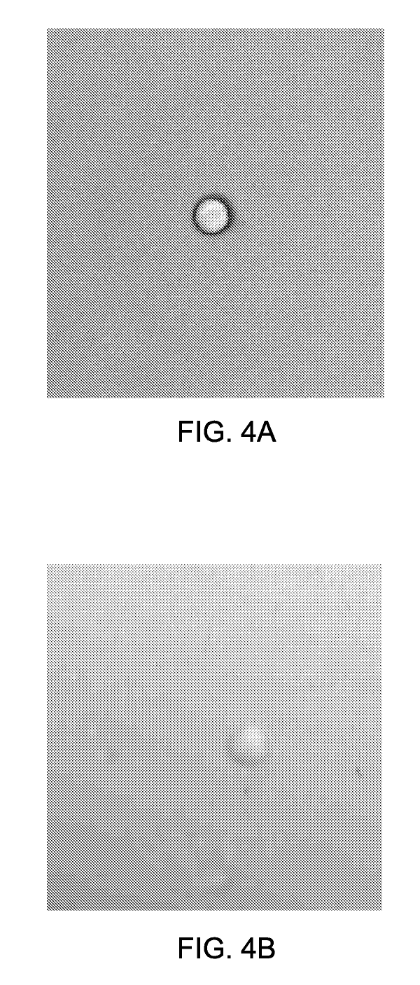

The amplification of pUC 18 DNA using random primer and specific-primer capture beads resulted in bead-bound DNA. The amplification of captured DNA present on a single bead coated with attached specific primers produced a bead completely coated with amplified DNA as shown in FIG. 4A. FIG. 4B shows the same bead isolated from a mixture containing many such beads, demonstrating that individual bead can be isolated from such a mixture.

### Example 6

### Restriction Digestion

The amplified product from NTC, and the amplified product from pUC 18 plasmid DNA were captured on the specific primer coated beads as described above. The beads were washed 3 times with TE and then were subjected to EcoRI restriction digestion. The pUC 18 DNA (as a positive control) was separately subjected to restriction digestion. The DNA was digested by adding 10 units of EcoRI, 1 μL 10× Enzyme buffer, 1 μl TE-washed beads, and 7 μL water to a total reaction volume of 10 μl and the reaction mixture was incubated in a water bath at 37° C. for 1 hour.

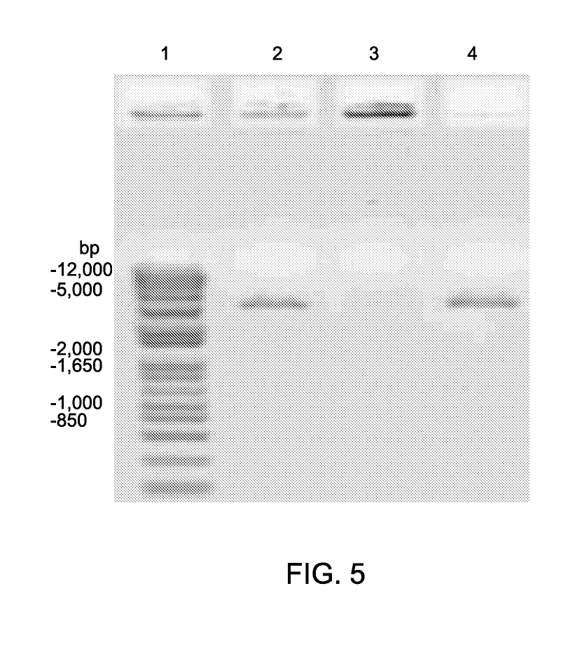

The restriction digestion products were loaded on to an agarose gel for analyzing the molecular weight of the digested product with respect to the standard molecular weight marker (lane 1) as shown in FIG. 5. The amplified pUC18 DNA was captured by beads followed by EcoRI restriction digestion, and the digested product was loaded on to lane 2 of the agarose gel (as demonstrated in FIG. 5). The purified pUC18 plasmid DNA was digested by EcoRI and was loaded on to lane 4 as a control, which shows the same molecular weight for the restriction digestion product DNA of pure pUC 18 and the amplified bead-bound pUC 18 DNA after restriction digestion. The restriction digestion product of NTC amplified DNA was loaded in lane 3, which shows the absence of background amplification product captured by beads. This demonstrates the simultaneous amplification and capture of target DNA without background amplification using the method described above.

### Example 7

### Transfer of DNA from One Bead to Other Beads

For estimating DNA amplification, capture and transfer of DNA from one bead to another, the following method was performed. A DNA amplification reaction was initiated by incubating the de-contaminated primer-nucleotide mix, the de-contaminated enzyme mix along with primer-coated capture beads and with pUC18 plasmid DNA template at 30° C. for about 400 min in a tube. The amplification reaction mixture was the same as described above. The amplified DNA was captured on beads present in a tube.

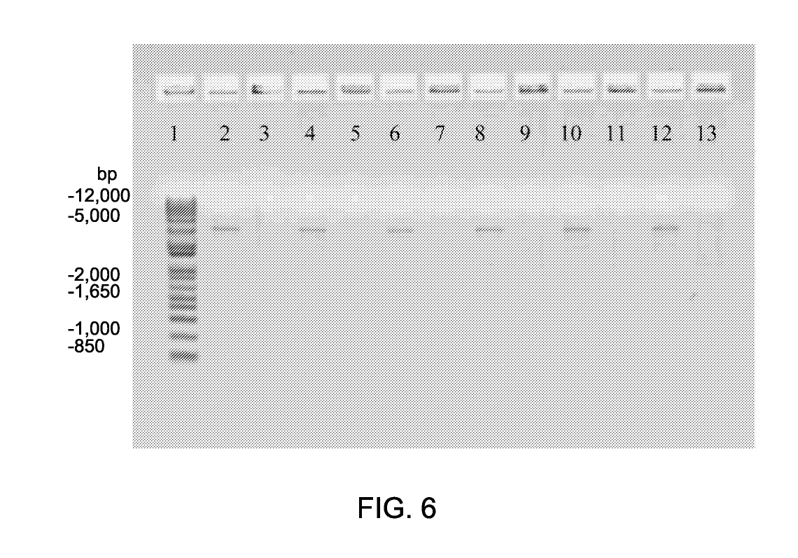

Five individually isolated beads with captured DNA were transferred to separate reaction tubes each, as described above. The excess TE was removed from each tube, and 10 μL of amplification reaction mixture was added to each bead. The amplification reaction mixture was composed of 40 μM primer (partially constrained primer of sequence, +W+WNN*S in which W denotes either A or T/U, and S denotes either G or C, N represents a random nucleotide (i.e., N may be any of A, C, G, or T/U) a plus (+) sign preceding a letter designation denotes that the nucleotide designated by the letter is a LNA nucleotide, a star (*) sign preceding a letter denotes that the nucleotide designated by the letter is a phosphorothioate modified nucleotide.); 400 μM dNTPs (400 μM each of dATP, dCTP, dGTP, dTTP), and 200 ng phi29 DNA polymerase (200 ng per 10 μL reaction). The incubation was performed in 50 mM HEPES buffer (pH=8.0) containing 15 mM KCl, 20 mM MgCl₂, 0.01% (v/v) Tween-20, 1 mM TCEP. The reactions were allowed to incubate at 30° C. for about 400 min. After amplification of the DNA that had been captured on these individual beads, 1 μL of the amplification reaction mixture supernate was removed, leaving the bead in the tube. The bead-bound DNA was digested by restriction enzyme EcoRI. The EcoRI digestion products for different tubes were loaded on an agarose gel as shown in FIG. 6, lanes 2, 4, 6, 8, and 10 (5 separate beads were isolated and amplified DNA bound to it), whereas the same samples without digestion were loaded to the agarose gel as shown in FIG. 6, lanes 3, 5, 7, 9, and 11. The 2.7 KB amplification product from pUC18 can clearly be seen in each lane. This demonstrates that each individual bead that was added to the amplification reaction had indeed captured the pUC18 amplification product during the initial amplification reaction. This demonstrates a non-limiting example of how this method could be used to amplify and capture DNA.

In another reaction, one bead with captured amplified DNA was transferred to a tube already containing fresh amplification reaction mixture along with additional specific primer-coated beads. The amplification reaction mixture was composed of 40 μM primer (partially constrained primer of sequence, +W+WNN*S in which W denotes either A or T/U, and S denotes either G or C, N represents a random nucleotide (i.e., N may be any of A, C, G, or T/U) a plus (+) sign preceding a letter designation denotes that the nucleotide designated by the letter is a LNA nucleotide, a star (*) sign preceding a letter designation denotes that the nucleotide designated by the letter is a phosphorothioate modified nucleotide.); 3 μL specific primer (approximately 70,000 beads per microliter, specific primer is covalently attached to the beads); 400 μM dNTPs (400 μM each of dATP, dCTP, dGTP, dTTP); and 200 ng Phi29 DNA polymerase (200 ng per 10 μL reaction). The incubation was performed in 50 mM HEPES buffer (pH=8.0) containing 15 mM KCl, 20 mM MgCl₂, 0.01% (v/v) Tween-20, 1 mM TCEP. Here, the amplification reactions included DNA captured on a single bead, which had been transferred from the previous amplification reaction tube as described above. The amplified DNA from the single bead was amplified further in this reaction, and during the reaction was transferred to the other beads. The amplified DNA captured on the additional beads was subjected to 3 washes by TE as described above, and followed by restriction digestion by EcoRI. 1 μL of the resulting washed beads was removed and digested with EcoRI as described in example 6. The EcoRI digestion product was loaded to agarose gel as shown in FIG. 6, lanes 12, and the undigested product was loaded on lane 13. The above example clearly demonstrates that the DNA which had originally been amplified and captured on beads in the first amplification reaction was then subsequently amplified off on that single bead in the subsequent amplification reactions and captured on a population of new capture beads. In this example, the single pUC18-coated bead was used by this method to create approximately 150,000 additional pUC18-coated beads. This demonstration is a non-limiting example of how this method could be used to transfer DNA from one location or surface to another.

### Example 8

### Sequence Analysis

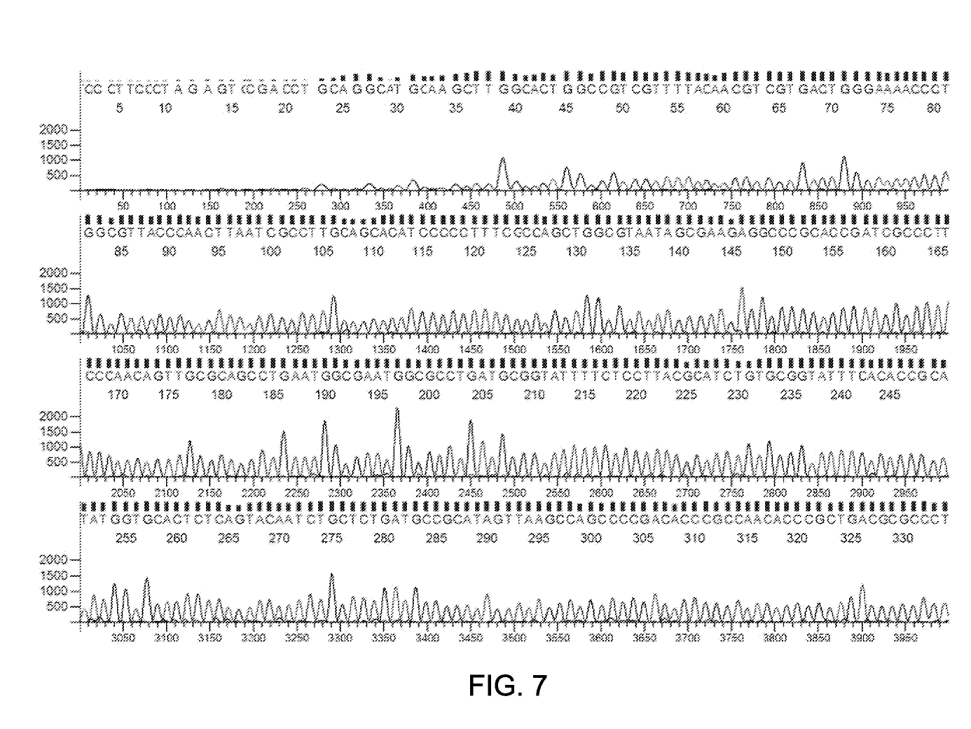

As a further demonstration that the beads described above contain amplified template, DNA sequence information was obtained from the beads. The amplified DNA was captured on beads as described for example 5 above. After amplification and capture, the TE-washed beads were used as a template for DNA sequencing reactions to demonstrate that the beads contained amplified DNA attached to the surface. 1 μL of the resulting washed beads (50,000 beads) were removed and 3.2 pmoles (1 μL) of M13 reverse sequencing primer, 4 μL Big Dye DNA sequencing premix, 2 μL sequencing buffer, and 12 μL water mixture was added to the said beads. The DNA was cycle-sequenced as per manufacturers recommendations, the sequencing products were purified by precipitation and resolved on an ABI 3730×1 sequencing machine (Applied Biosystem). The sequence data (SEQ ID NO. 2) shows in FIG. 7. The sequence obtained from these beads had a signal strength equivalent to that normally obtained with about 200 ng of pUC18 template DNA. This indicates that each bead could have had about 10 picograms of attached amplified pUC18 DNA. The sequence obtained was an exact match to the sequence obtained from pUC18 DNA, indicating that the DNA attached to the beads was amplified pUC18 DNA.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for amplifying a nucleic acid, comprising:
providing a nucleic acid template, a DNA polymerase, deoxyribonucleoside triphosphates, a primer comprising a randomized sequence, and a specific primer; and
amplifying the nucleic acid template under isothermal conditions to form an amplified nucleic acid sequence.

2. The method of claim 1, wherein the random primer comprises at least one modified nucleic acid base.

3. The method of claim 2, wherein the modified nucleic acid base increases the melting temperature of the primer comprising a randomized sequence.

4. The method of claim 2, wherein the modified nucleic acid base is selected from a locked nucleic acid base, a peptide nucleic acid base, or a ribonucleic acid base.

5. The method of claim 1, wherein the specific primer is attached to a first substrate.

6. The method of claim 5, wherein the first substrate is selected from a bead, a test tube, a multi-well plate, or a slide.

7. The method of claim 5, wherein a material of the first substrate is selected from polymer, glass, or metal.

8. The method of claim 5, further comprising capturing the amplified nucleic acid sequence by hybridization of the amplified nucleic acid sequence with the specific primer to form a first substrate-bound nucleic acid sequence.

9. The method of claim 8, further comprising extending a nucleic acid sequence from the hybridization site of the specific primer using the first substrate-bound nucleic acid sequence as a template.

10. The method of claim 8, further comprising amplifying the first substrate-bound nucleic acid sequence by a primer comprising a randomized sequence to form a second amplified nucleic acid sequence.

11. The method of claim 10, further comprising capturing the second amplified nucleic acid sequence by a second substrate by hybridization of the second amplified nucleic acid with a specific primer attached to the second substrate.

12. The method of claim 1, wherein the specific primer is attached to a capturing agent.

13. The method of claim 12, wherein the capturing agent comprises an affinity tag.

14. The method of claim 1, wherein the amplification is performed in an emulsion.

15. The method of claim 1, wherein the amplification comprises a rolling circle amplification or a multiple displacement amplification.

16. The method of claim 1, wherein the amplified nucleic acid sequence is a tandem repeat nucleic acid sequence.

17. The method of claim 1, wherein the nucleic acid template is a circular nucleic acid.

18. The method of claim 1, wherein the nucleic acid template comprises a recombination site.

19. The method of claim 1, wherein the nucleic acid template is a DNA.

20. The method of claim 1, wherein the DNA polymerase is a Phi 29 DNA polymerase.

21. The method of claim 1, wherein the random primer comprises at least 5 nucleotides.

22. A kit for amplifying a nucleic acid comprising:
a Phi29 DNA polymerase;
at least one primer comprising a randomized sequence; and
at least one specific primer.

23. The kit of claim 22, wherein the specific primer is attached to a substrate or a capturing agent.


SEQUENCE LISTING (2 sequences)

SEQ ID NO: 1 - DNA, 23 bp, Artificial Sequence
  Description of Artificial Sequence Synthetic oligonucleotide
gttttcccagtcacgacgttgta

SEQ ID NO: 2 - DNA, 333 bp, Artificial Sequence
  Description of Artificial Sequence Synthetic polynucleotide
cccttccctagagtccgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccct